(19) United States
(12) Patent Application Publication
(10) Pub. No.: US 2012/0288926 A1
(43) Pub. Date: Nov. 15, 2012

(54) Blood Cell Trajectory Displaying Device

(75) Inventor: Takanori Murayama, Hino-shi (JP)

(73) Assignee: Konica Minolta Advanced Layers, Inc., Hachioji-shi, Tokyo (JP)

(21) Appl. No.: 13/511,605

(22) PCT Filed: Oct. 27, 2010
(86) PCT No.: PCT/JP2010/069051

§ 371 Date: May 23, 2012

(30) Foreign Application Priority Data
Nov. 26, 2009 (JP) 2009-268503

(51) Int. Cl. C12M 1/34

(52) U.S. Cl. 435/288.7

(57) ABSTRACT

Disclosed is a blood cell trajectory display device (1) for displaying the blood cell trajectory leading to an agglutination, that is equipped with a TV camera (3) for sequential photographing the flow of blood, a calculation unit (70) that analyzes the blood flow images obtained by the TV camera (3), and a display (8). The calculation unit (70) analyzes a plurality of blood flow image frames and detects the agglutination of blood cells in the blood, and in cases in which blood cell agglutination has been detected, detects the position of said blood cells in the blood flow image frame prior to the frame in which the agglutination was detected, and requests the trajectory of said blood cells to the agglutination point. The display (8) displays the blood cell trajectory requested by the calculation unit (70).

### FIELD OF THE INVENTION

The present invention relates to a blood cell trajectory displaying apparatus which is displaying trajectory of a blood cell.

### BACKGROUND OF THE INVENTION

With increasing interest in health in recent years, particular importance has come to be given to the blood fluidity as a health barometer. One of the ways of checking the blood fluidity disclosed so far is a technique for measuring the time required for blood to run through a micro channel array having a plurality of microscopically small channels (see Patent Literature 1 for example).

Incidentally, the blood of lower fluidity tends to be occurred to agglutination where blood cell is retained to be combined into conglomerates (see FIG. 13). Since occurring of agglutination influences the blood fluidity greatly, the soundness of the state of blood fluidity can be judged by detecting the occurring of agglutination and explaining the trajectory of a blood cell which result in the agglutination. Therefore, it is desired a technology which displays the trajectory of a blood cell result in the agglutination.

### EARLIER TECHNOLOGICAL LITERATURE

### Patent Literature

Patent Literature 1: Japanese Unexamined Patent Application Publication No. 2006-145345

### SUMMARY OF THE INVENTION

### Problems to be Solved by the Invention

The problem of the present invention is providing a blood cell trajectory displaying apparatus which displays the trajectory of a blood cell until it agglutinate.

### Means for Solving the Problems

To solve the above mentioned problems, the invention of Claim 1 is a blood cell trajectory displaying apparatus comprising:

a photographing device for sequential photographing blood flow images;

an analysis device for detecting an agglutination of a blood cell in blood by analyzing the blood flow images of a plurality of frames taken by the photographing device, for detecting a position of the blood cell in the blood flow image of a previous frame of a frame of which the agglutination is detected when the agglutination of the blood cell is detected, and for obtaining a trajectory of the blood cell to a position of the agglutination; and

a display device for displaying the trajectory of the blood cell obtained by the analysis device.

The invention of Claim 2 is the blood cell trajectory displaying apparatus described in Claim 1 further comprises a memory device for memorizing blood flow images of a predetermined number of previous and subsequent frames of the frame of which the agglutination is detected, when the analysis device detects the agglutination of the blood cell.

The invention of Claim 3 is the blood cell trajectory displaying apparatus described in Claims 1 or 2,

wherein the display device displays a shape of the blood cell in the blood flow image of the frame of which the agglutination of the blood cell is detected and a shape of the blood cell of a previous frame of the aforementioned frame.

The invention of Claim 4 is the blood cell trajectory displaying apparatus described in any one of Claims 1 to 3,

wherein the analysis device calculates a change of area of the blood cell along the trajectory of the blood cell to a position of the agglutination by calculating an area of the blood cell in the blood flow image of the frame of which the agglutination of the blood cell is detected and an area of the blood cell of a previous frame of the aforementioned frame, when the analysis device detects the agglutination of the blood cell.

The invention of Claim 5 is the blood cell trajectory displaying apparatus described in any one of Claims 1 or 4,

wherein the analysis device calculates at least one of a movement speed and a movement angle between frames of the blood cell in the blood flow image of the frame of which the agglutination of the blood cell is detected and a previous frame of the aforementioned frame, when analysis device detects the agglutination of the blood cell.

The invention of Claim 6 is the blood cell trajectory displaying apparatus described in Claim 5,

wherein the analysis device for determining that the blood cell is abnormal when at least one of the calculated movement speed and the movement angle exceeds predetermined ranges; and

the display device emphasizes and displays the trajectory of the blood cell, when the blood cell is determined as an abnormal by the analysis device more than the trajectory of the blood cell which is not determined as an abnormal.

### Objects of the Invention

According to the invention of Claim 1, it is provided with: the agglutination of the blood cell in the blood is detected by analyzing the blood flow images of a plurality of frames, and when the agglutination of the blood cell is detected, the trajectory of the blood cell to a position of the agglutination is obtained by detecting a position of the blood cell in a previous frame of a frame of which the agglutination of the blood cell is detected. And, this trajectory of the blood cell is displayed. Therefore, the trajectory of the blood cell which results in the agglutination can be displayed, and on the basis of this, the soundness of the state of blood fluidity can be determined easily visually.

According to the invention of Claim 2, since the blood flow images of a predetermined number of previous and subsequent frames of the frame of which the agglutination is detected are memorized when the agglutination of the blood cell is detected, that is, the blood flow images related to occurring agglutination is memorized, it is not necessary to memorize blood flow images other than this. Therefore, large capacity for a memory device is not required but reduction of cost of the apparatus can be aimed at.

According to the invention of Claim 3, since a shape of the blood cell in the blood flow image of the frames of which the agglutination of the blood cell is detected and a shape of the blood cell of a previous frame of the aforementioned frame is displayed, the change of shape of a blood cell along the trajectory of the blood cell to a position of the agglutination is displayed. Therefore a shape change of the blood cell which results in the agglutination, and the ease of changing of the blood cell can be recognized visually, and the soundness of a flow state of blood can be visually determined easily based on this.

According to the invention of Claim 4, since area change of the blood cell along with the trajectory of the blood cell to the agglutination point is computed, the ease of changing of shape of the blood cell which results in agglutination is expressed quantitatively. Therefore, the soundness of a flow state of blood can be determined by comparing area change of the above mentioned blood cell with area change of the blood cell in healthy blood, for example etc.

According to the invention of Claim 5, the trend of the blood cell which results in agglutination is expressed quantitatively by computing at least one of a movement speed and a movement angle between frames of the blood cell in the blood flow image of the frame of which the agglutination of the blood cell is detected and a previous frame of the aforementioned frame. Therefore, the soundness of a flow state of blood can be determined by comparing at least one of the movement speed and the movement angle of the blood cell concerned with the value of the blood cell in healthy blood, for example etc.

According to the invention of claim 6, the blood cell is determined as an abnormal when at least one of the movement speed and the movement angle exceeds predetermined ranges, and when the blood cell is determined as an abnormal, since the trajectory of the blood cell is emphasized and displayed more than the trajectory of the blood cell which is not determined as an abnormal, abnormalities of a trend of a blood cell can be recognized visually. Therefore, the soundness of a flow state of blood can be determined easily.

### BRIEF DESCRIPTION OF THE DRAWINGS

FIG. 1 is a block diagram showing the overall structure of a blood cell trajectory displaying apparatus.

FIG. 2a is a top view of a microchip.

FIG. 2b is a side view of a microchip.

FIG. 3 is a partial enlarged view of a microchip.

FIG. 4a is a diagram for explaining gates of a microchip.

FIG. 4b is a diagram for explaining gates of a microchip.

FIG. 5 is a flow chart showing the display of the trajectory of the blood cell by a blood cell trajectory displaying apparatus.

FIG. 6 is a diagram showing an example of an image in which the gate and the front and back field of the gate are divided in grid pattern.

FIG. 7a is an example of a two-dimensional speed map.

FIG. 7b is an example of a two-dimensional speed map.

FIG. 7c is an example of a two-dimensional speed map.

FIG. 8 is a diagram showing a pattern matching performed tracking back frames.

FIG. 9 is a diagram showing an example of an image which displayed the trajectory of the blood cell result in the agglutinated by the arrow.

FIG. 10 is a diagram showing an example of an image which displayed the trajectory of the blood cell result in the agglutinated by the blood cell itself.

FIG. 11 is a diagram showing an example of an image of the shape of the blood cell displayed aside from the trajectory of the blood cell until agglutinated.

FIG. 12 is a diagram showing an example of a blood flow image which is determined as an abnormal.

FIG. 13 is a diagram showing an example of an blood flow image which the agglutination is occurring.

### DESCRIPTION OF THE PREFERRED EMBODIMENT

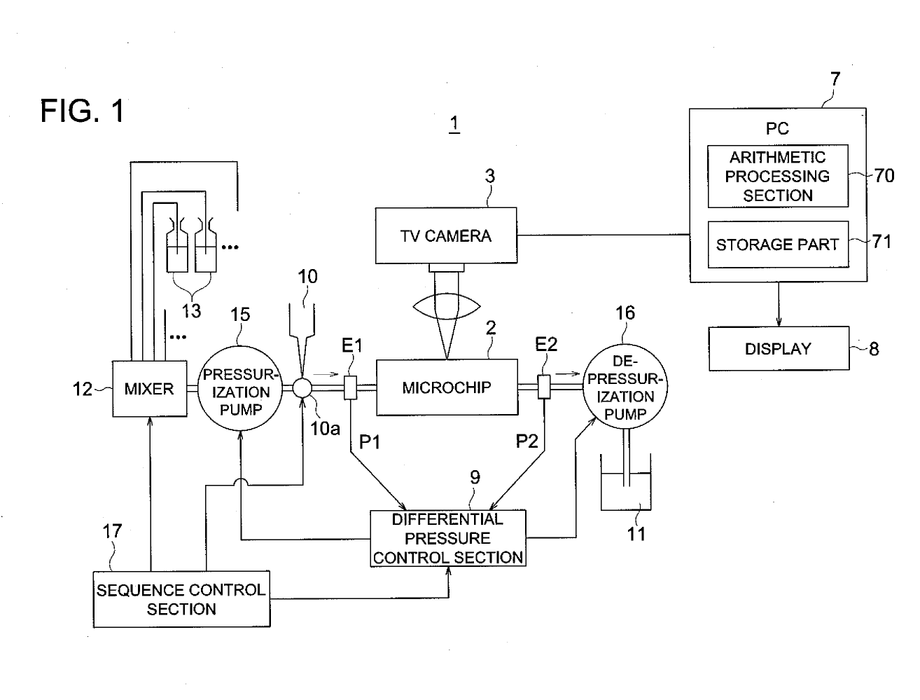

The following describes the embodiments of the present invention with reference to figures. FIG. 1 is a block diagram showing the overall structure of a blood cell trajectory displaying apparatus 1 concerning the present invention.

As shown in FIG. 1, the blood cell trajectory displaying apparatus 1 leads the blood supplied from an inlet 10 to the discharge tank 11 through a microchip 2, and detects the agglutination of the blood based on the information obtained in this process, displays trajectory of the blood cell to the position of the agglutination. In this embodiment, the agglutination indicates the conglomerate composed of the stagnated blood cell.

Specifically, the blood cell trajectory displaying apparatus 1 is provided a microchip 2, a TV camera 3 for taking an image of the flow of blood in the microchip 2, a personal computer (PC) 7 for detecting the agglutination by analyzing an blood flow image taken by the TV camera 3, display 8 for displaying the blood flow images etc., and a differential pressure control section 9 for controlling the blood flow in the microchip 2.

The blood cell trajectory displaying apparatus 1 is thither provided with a plurality of solution bottles 13 which communicate with a blood flow path through a mixer 12 so that such a liquid as physiological saline solution or physiological active substance can be mixed with blood and led to the microchip 2. When the differential pressure control section 9 controls to adjust the differential pressures of the front and the back the microchip 2, a desired amount of the blood mixed with such a liquid as physiological saline solution or physiological activated substance (hereinafter referred to as “blood”) flows through the microchip 2. Further, the differential pressure control section 9, the mixer 12, and valve 10a of the inlet 10 are integrally controlled by a sequence control section 17.

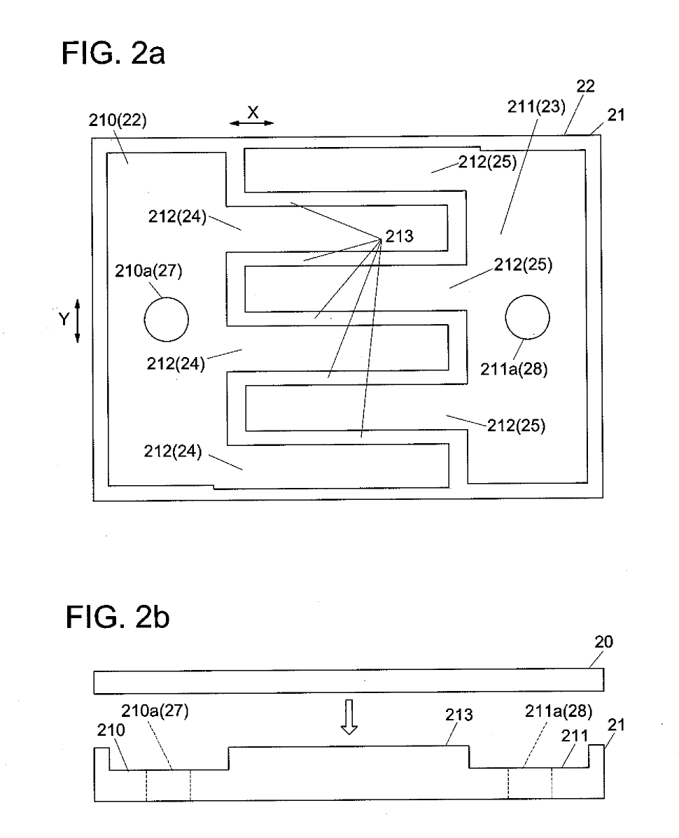

FIG. 2(a) is a top view of a micro chip 2, and FIG. 2(b) is a side view.

As shown in FIG. 2, the microchip 2 is formed by piling up a rectangle shape glass plate 20 and a base plate 21. Glass plate 20 is formed as a flat plate shape and covers inner surface (upper surface in FIG. 2(b)) of the base plate 21.

The base plate 21 is provided with concavity parts 210, 211 in both ends, and a plurality of groove parts 212 between the concavity parts 210, 211.

Among these, a concavity part 210 has a penetration port 210a connecting to the inlet 10 and forming an inflow port 27 of blood on the bottom, and an upper stream side storing section 22 which stores blood is formed between glass plates 20.

Similarly, the concavity part 211 has a penetration port 211 a connecting to a discharge port 21b and forming an outflow port 28 of blood on the bottom, and a lower stream side storing section 23 which stores blood is formed between glass plates 20.

A plurality of the grooves 212 is arranged as extend in parallel to the direction (the direction X in figures) which connects concavity part 210 and concavity part 211, and is divided in the direction (the direction of Y in figures) which intersects perpendicularly in the direction X by terrace section 213 which is arranged as extend in the direction X. These plurality grooves 212 are connected alternately to the concavity part 210 or concavity part 211, and by this, upper stream side blood circuits 24 which blood is made to flow from upper stream side storing section 22 and lower stream side blood circuits 25 which blood is made to flow into lower stream side storing section 23 are formed between glass plates 20.

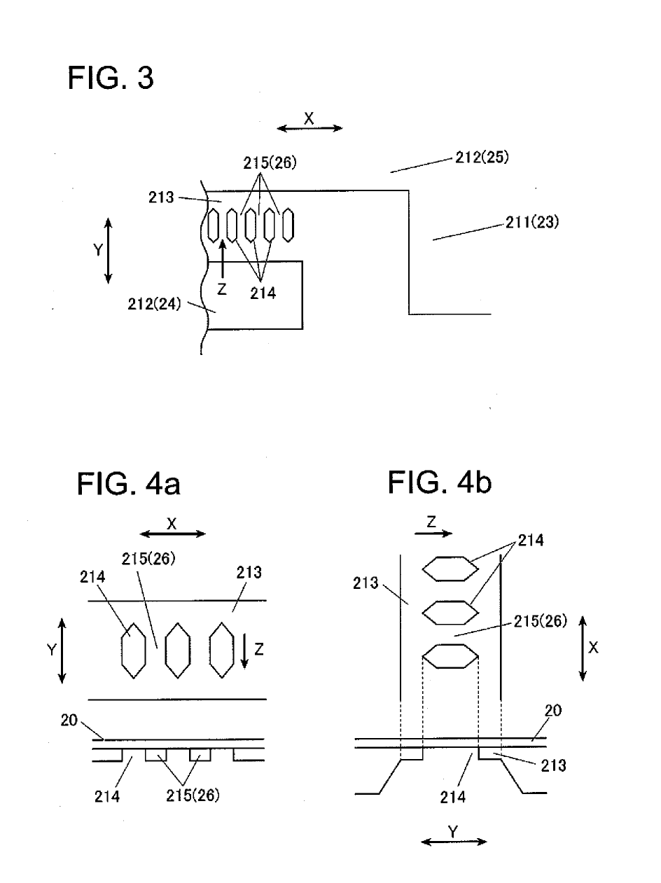

FIG. 3a is a partial enlarged view of a microchip 2, FIGS. 4a and 4b are diagram for explaining gates 26 mentioned later. Both upper figures of FIGS. 4a and 4b are top views of a terrace section 213, and lower figures of FIGS. 4a and 4b are sectional side views of that.

As shown in these figures, a plurality of hexagonal bank parts 214 is arranged at an upper end part of the terrace section 213 in the direction X, and contacts with glass plate 20 by the top plane.

These plurality bank parts 214 form interval part 215 among each other. The interval part 215 constitutes a gate 26 between the undersurfaces of glass plate 20, as a detailed channel in which blood flows in the direction (the direction of Z in figures) parallel to the direction of Y. Although limitation in particular is not carried out, the section shape of gate 26 is making the flat rectangle according to the shape of red blood cells (disk shape where middle became depressed and a section is flat elliptical shape.), and the size of the section of this gate 26 is smaller than the size of red cells. The state of passing the blood vessel, such as a capillary vessel while making red cells change own shape is observable, and, degree of smoothness of blood flow in the inside of a blood vessel is reproducible in imitation.

With microchip 2 providing aforementioned composition, blood led from the inlet 10 is stored in the upper stream side storing section 22, after passes through the gate 26 and the lower stream side blood circuits 25 from the upper stream side blood circuits 24, stored in the lower stream side storing section 23, and discharged to the discharge tank 11. In this process, the blood cell in the blood which flows through gate 26, for example, red blood cells pass the inside of this gate 26 changing own shape.

In addition, the pressure sensors E1 and pressure sensors E2 which measure the pressure of the blood among an entrance and an exit of the microchip 2 are provided in an upper stream and a lower stream of the microchip 2. These pressure sensors E1 and pressure sensors E2 output the measured pressure P1 in the upper stream of the chip and pressure P2 in the lower stream of the chip to the differential pressure control section 9.

As shown in FIG. 1, the TV camera 3 is provided for countering to the glass plate 20, and the blood flow pass through the gate 26 is captured through the glass plate 20. The TV camera 3 is a digital CCD camera, for example, is a high-speed camera for capturing a blood flow image sequential, and is a camera capable of capturing a moving image. The blood flow image captured by the TV camera 3 is outputted to the personal computer 7 and is displayed by display 8.

The personal computer 7 is provided an arithmetic processing section 70 and a storage part 71. Among these, the arithmetic processing section 70 detects the agglutination of the blood cell of the blood and obtains the trajectory of the blood cell to a position of the agglutination by analyzing the blood flow images captured by the TV camera 3. In addition to this, the arithmetic processing section 70 computes various values mentioned later. A storage part 71 memorizes blood flow images of a predetermined number of previous and subsequent frames of the frame of which the agglutination is detected, when the arithmetic processing section 70 detects the agglutination of the blood cell.

A display 8 displays the trajectory of the blood cell obtained by the arithmetic processing section 70, and displays a shape of the blood cell which changes along with the trajectory the aforementioned blood cell. In addition to this, the display 8 is possible to display blood flow images which outputted by the TV camera 3, the calculation result which personal computer 7 computed, etc.

The differential pressure control section 9 controls differential pressure the front and the back the microchip 2 according to the control instruction from a sequence control section 17. Specifically, the differential pressure control section 9 controls pressurization pump 15 arranged in the upper stream of the microchip 2 and depressurization pump 16 arranged in the down stream of the microchip 2, respectively, so that the pressure P1 in the upper stream of the chip and pressure P2 in the lower stream of the chip turn into predetermined pressure. This differential pressure control section 9 and sequence control section 17 can be constituted with personal computer 7.

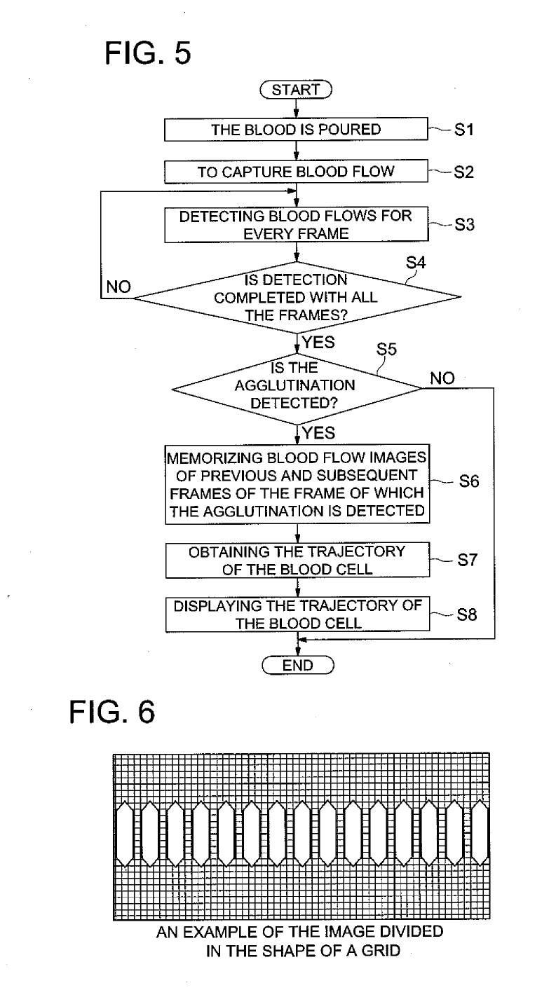

The following describes the operation of the blood cell trajectory displaying apparatus 1 when displaying the trajectory of the blood cell. FIG. 5 is a flow chart showing the display of the trajectory of the blood cell by the blood cell trajectory displaying apparatus 1.

As shown in this figure, the blood to be measured is poured to the microchip 2 (Step S1). Specifically, the blood to be measured is poured to the inlet 10, and physiological saline solution is supplied to the solution bottle 13, as required. Then a predetermined differential pressure is applied to the microchip 2 by the differential pressure control section 9 so that blood is poured to the microchip 2.

Next, the blood flow passing through the gate 26 is captured sequentially by the TV camera 3 (Step S2). At this time, it is enough if the capturing area of TV camera 3 includes either one of plurality gates 26, and the field of terrace section 213 the front and the back the gate 26. And images are captured until all the blood has poured through microchip 2.

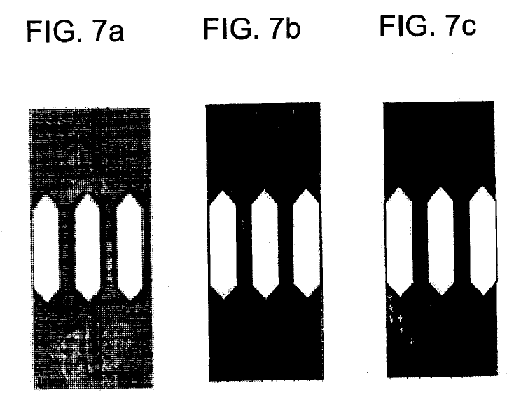

Next, agglutination of a blood cell is detected from the captured blood flow images of a plurality of frames (Step S3). This step is performed when arithmetic processing section 70 of personal computer 7 analyzes the blood flow images of a plurality of frames captured in Step S2 for every frame in order of the captured time. A publicly known method, for example described to Japanese Patent Application Publication No. 2006-223761 etc., can be used for detection of the agglutination. In more detail, the gate 26 and the field of front and back of the gate 26 in the blood flow image is divided in the shape of a grid as shown in FIG. 6 and rate vector of a blood cell is computed for every grid. FIGS. 7(a) to 7(c) show examples of images of a two-dimensional speed maps which are drawn by stacking the computed rate vector and the blood flow image. And, a field of a grid where the rate vector is not computed among this two-dimensional speed map (a field where the arrow of the rate vector is not drawn in FIGS. 7(a) to 7(c)) is detectable as a field in which the blood cell is stagnated, that is a field in which the blood agglutination occurred. As for the grid formation on the blood flow image, it is preferred uniting the width of a grid with the width of gate 26 so that at least one grid may be formed in gate 26.

Next, as shown in FIG. 5, the arithmetic processing section 70 determines whether detection of agglutination on the blood flow image of all the frames was completed (Step S4), and when there is a frame on which detection is not performed (Step S4; No), shifts to above mentioned step S3, and detects the agglutination to the blood flow image of the frame concerned.

When detection of agglutination is completed to the blood flow image of all the frames, (Step S4; Yes) the arithmetic processing section 70 determines whether an agglutination was detected from any one of frames in above mentioned step S3, (Step 55). And when agglutination is detected from no frames in Step S3, (Step S5; No) the blood cell trajectory displaying apparatus 1 ends operation of display of a trajectory of a blood cell.

On the other hand, when agglutination is detected from one or some of the frames (Step S5; Yes)., the arithmetic processing section 70 memorizes blood flow images of a predetermined number of previous and subsequent frames of the frame of which the agglutination is detected into a storage part 71 (Step S6) With this embodiment, the arithmetic processing section 70 memorizes the blood flow images of which agglutination was detected and 50 frames to each previous and subsequent form the frame. When agglutination is detected by the same grid in a different frame at this time, the frame captured most early is determined as the frame which agglutination is detected. “Previous and subsequent of the frame,” it is a meaning previous and subsequent of the flames in the order of the captured time, and “the previous frame” in the following explanation is the similar meaning.

Next, the arithmetic processing section 70 obtains the trajectory of the blood cell to the agglutination point which agglutination is occurred (Step S7). And, “agglutination point” is a point which the blood cell conglomerates in the capturing field of TV camera 3.

In this step, first, arithmetic processing section 70 processes the blood flow image of the frame of which agglutination was detected in Step S3, and identifies each blood cell from the field in which the blood cell stagnated (henceforth a stagnated field). Specifically, the edge of each blood cell can be emphasized and identified by filtering with Sobel filter over vertical and horizontal both directions to the blood flow image, for example. When blood cell kinds differ, it can identify using hue or a size. For example, red blood cells are identifiable as an image region in a red hue range. White blood cells can also be identified using luminosity and can also be identified as an image region with few edges per unit area using being larger than other blood cell kinds. In addition, besides these identifying methods, the publicly known method of a statement can be used for Japanese Unexamined Patent Application Publication No 10-48120A, Japanese Unexamined Patent Application Publication No 10-90163A, Japanese Unexamined Patent Application Publication No 10-274652A, etc., for example, and a blood cell kind can be identified.

And, the arithmetic processing section 70 reads the blood flow image of 50 frames previous the frame which agglutination was detected from storage part 71, and identifies each blood cell to the blood flow image concerned similarly.

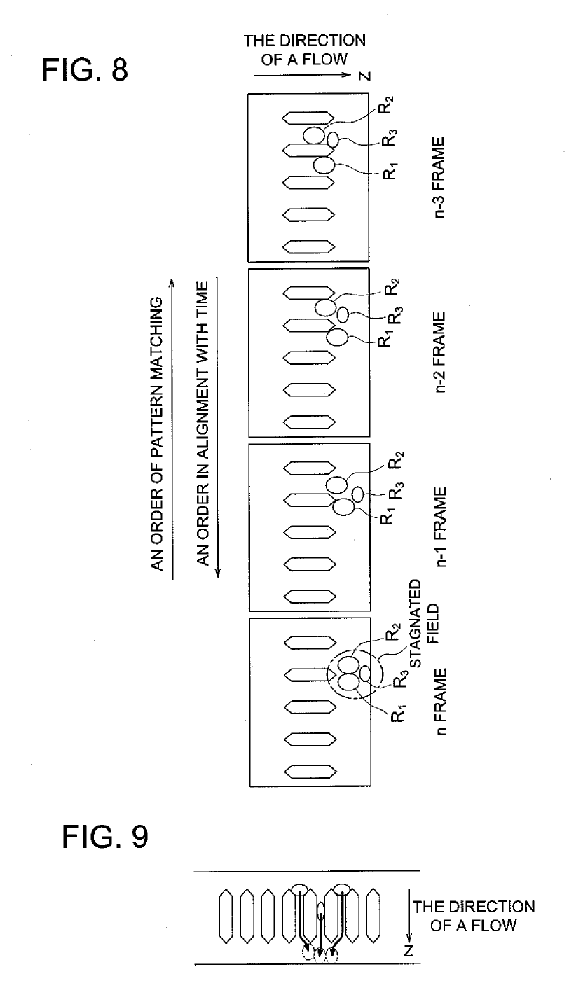

And the arithmetic processing section 70 goes back the order of time flow the frame as which agglutination was detected, and tracing back frames, and detects the position of the blood cell which forms the stagnated field in the frame of which agglutination was detected in the blood flow image of these each frame. In more detail, when the frame of which agglutination was detected is made into n-th (n frame) frame in the order of time, and three blood cells R1, R2, and R3 form a stagnant field in this n frame, the position of blood cells R1, R2, and R3 in each frame are detected by performing pattern matching, going back with n−1 frame, n−2 flame, . . . from then frame, as shown in FIG. 8. This pattern matching is performed to 50 frames in which each blood cell has identified. However, pattern matching and identification of a blood cell is performable in parallel.

In this way, the trajectory of the blood cell to an agglutination point is obtained by connecting the position of the blood cell of detected 50 frames.

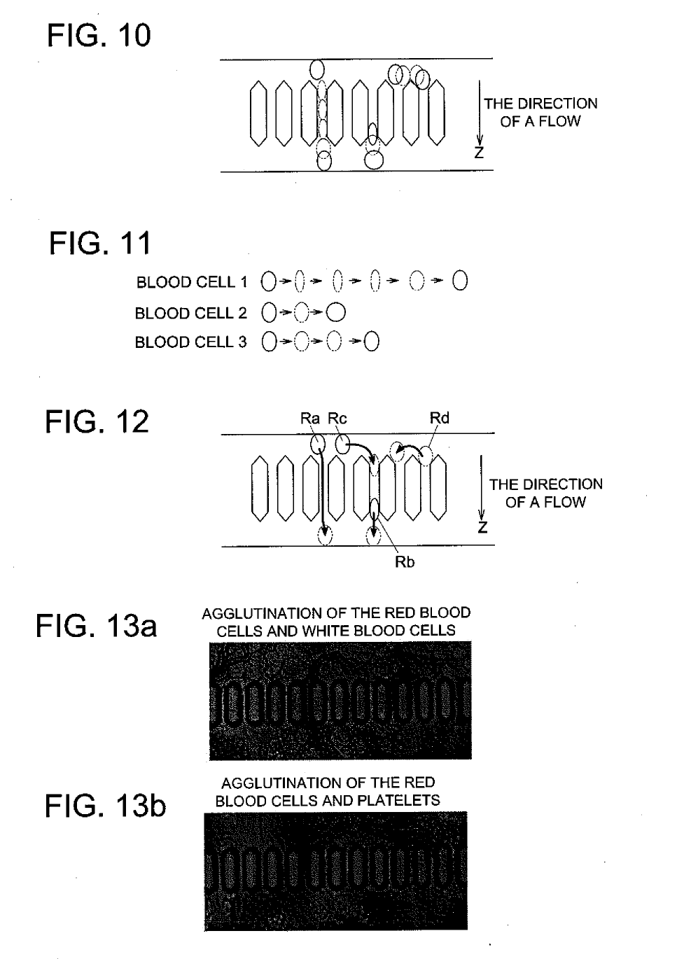

Next, the trajectory of the blood cell obtained at Step S7 is displayed on display 8 (Step S8). At this time, it is useful even if the trajectory of a blood cell is displayed by the arrow on the image of a flow path of blood cells as shown in FIG. 9, and by the image of a plurality of the blood cells themselves instead of the arrow on the image as shown in FIG. 10. Shape change of the blood cell in alignment with the trajectory of the blood cell to an agglutination point can be shown by displaying the shape of the blood cells in the blood flow images of the frames of which the agglutination of the blood cell is detected and a previous frames of the aforementioned frame. However, as shown in FIG. 11, only the image of a blood cell may be independently displayed from the trajectory of a blood cell. Although figure is omitted, it may display an arrow and the image of a blood cell simultaneously on the image of the flow path, and may display the animation of a blood cell.

At this time, it is preferred to compute the many amounts relating to the agglutination with the display of the trajectory of a blood cell. An area change (volume change) of the blood cell which result in agglutination, movement speed and a movement angle are mentioned as this amounts relating to the agglutination.

Among these, in calculation of area change of a blood cell, the area of the blood cell in the blood flow image of the flame of which the agglutination of the blood cell is detected and an area of the blood cell of a previous frame of the aforementioned frame, is computed by the arithmetic processing section 70. And the area change of the blood cell concerned in alignment with the trajectory of the blood cell to a agglutination point is computed by calculating the amount of change which met in order of the time of the frame about the computed area.

Calculation of the movement speed and a movement angle of the blood cell are preformed by the arithmetic processing section 70 by analyzing the blood flow image of the frame of which the agglutination of the blood cell is detected and an area of the blood cell of a previous frame of the aforementioned frame. In more detail, the movement speed can be computed from such migration length of the blood cell between frames and shutter speed, and the movement angle can be computed as an angle which the move direction of a blood cell and a certain reference direction (for example, the direction of Z which is the direction of a flow of a blood cell) make.

Here, based on the movement speed and movement speed of a blood cell which were computed, it can be determined whether the trend of the blood cell is abnormal. Specifically, when at least one of the computed movement speed and a movement angle of the blood cell exceeds the predetermined range, the arithmetic processing section 70 determines that the blood cell is abnormal. For example, the case where exceeding plus or minus 30% of the average speed of the blood cells which are not agglutinate, or the movement angle of plus or minus 20deg to the direction of Z etc. are these predetermined ranges. And when it is determined that a blood cell is abnormal, as shown in FIG. 12, the trajectory (arrow) of the blood cell is emphasized rather than the trajectory (for example, arrow shown in FIG. 9) of the blood cell which is not determined as an abnormal, and is displayed on display 8. Here, FIG. 12 shows blood cell Ra which movement speed until agglutination is quicker than predetermined range, blood cell Rb which movement speed until agglutination is slower than the predetermined range, and blood cell Rc and Rd with the movement angle until agglutination are larger than the predetermined range. As a embodiment which indicates the trajectory of a blood cell by emphasis, as shown in FIG. 12,

an arrow is made thick, and also the color of an arrow may be changed or it may be made to blink. When using the image of the blood cell instead of an arrow as a trajectory of a blood cell, it can indicate by emphasis similarly.

According to the above blood cell trajectory displaying apparatus 1, while agglutination of the blood cell in blood is detected by analyzing a plurality of the frames of the blood flow image, when agglutination of a blood cell is detected, the trajectory of the blood cell to a agglutination point is obtained by detecting a position of the blood cell in the blood flow image of a previous frame of the frame of which the agglutination is detected. And the trajectory of this blood cell is displayed. Therefore, the trajectory of the blood cell which results in agglutination can be displayed, and the soundness of a flow state of blood can be visually determined easily based on this.

When agglutination of a blood cell is detected, since the blood flow image of a predetermined frame number is memorized previous and subsequent the frame of which agglutination is detected, that is, the blood flow image in comparison and strongly related to occurring of agglutination is memorized, and blood flow image other than this are not memorized. Therefore, storage part 71 does not need big capacity, but reduction of cost of the apparatus can be aimed at.

And since a shape change of the blood cell in the blood flow image of a previous frame of which the agglutination is detected, a shape change of the blood cell along with the trajectory of the blood cell to the agglutination point is displayed. Therefore a shape change of the blood cell to the agglutination, and by extension, the ease of changing of the blood cell can be recognized visually, and the soundness of a flow state of blood can be visually determined easily based on this.

Since area change of the blood cell along with the trajectory of the blood cell to the agglutination point is computed, the ease of changing of shape of the blood cell which results in agglutination is expressed quantitatively. Therefore, the soundness of a flow state of blood can be determined by comparing area change of the above mentioned blood cell with area change of the blood cell in healthy blood, for example etc.

The trend of the blood cell which results in agglutination is expressed quantitatively by computing at least one of the movement speed and the movement angle of the blood cell concerned between the blood flow image of the frames of which the agglutination of the blood cell is detected and a previous frame of the aforementioned frame. Therefore, the soundness of a flow state of blood can be determined by comparing at least one of the movement speed and the movement angle of the blood cell concerned with the value of the blood cell in healthy blood, for example etc.

The blood cell is determined as an abnormal when at least one of the movement speed and the movement angle exceeds predetermined ranges, and when the blood cell is determined as an abnormal, since the trajectory of the blood cell is emphasized and displayed more than the trajectory of the blood cell which is not determined as an abnormal, abnormalities of a trend of a blood cell can be recognized visually. Therefore, the soundness of a flow state of blood can be determined

The present invention should not be interpreted with limitation to the above mentioned embodiment, but, of course, change and improvement are possible suitably.

For example, although the above mentioned embodiment is given and explained blood as a sample, what is necessary is just a fluid sample which is not limited to blood but contains a material ingredient.

As for the frame number which goes back when obtaining for the trajectory of a blood cell, it is preferred not to be limited to 50 frames but for it to be able to change arbitrarily. When this frame number is changed, the predetermined frame number memorized by storage part 71 is changed similarly.

The frames do not need to be continuing even if the frames which goes back when obtaining for the trajectory of a blood cell, and the frames referred to when displaying the shape of a blood cell are plurality.

For example, when photography is captured by a high frame rate, the frame thinned out if needed may be used.

### DESCRIPTION OF SYMBOLS

1. blood cell trajectory displaying apparatus

3. TV camera (photographing means)

7. Personal computer

8. Display (display means)

70. Arithmetic processing section (arithmetic processing means)

71. Storage part (memory means)

1. A blood cell trajectory displaying apparatus comprising:
a photographing device for sequential photographing blood flow images;
an analysis device for detecting an agglutination of a blood cell in blood by analyzing the blood flow images of a plurality of frames taken by the photographing device, for detecting a position of the blood cell in the blood flow image of a previous frame of a frame of which the agglutination is detected when the agglutination of the blood cell is detected, and for obtaining a trajectory of the blood cell to a position of the agglutination; and
a display device for displaying the trajectory of the blood cell obtained by the analysis device.

2. The blood cell trajectory displaying apparatus of claim 1 further comprises a memory device for memorizing blood flow images of a predetermined number of previous and subsequent frames of the frame of which the agglutination is detected, when the analysis device detects the agglutination of the blood cell.

3. The blood cell trajectory displaying apparatus of claim 1,
wherein the display device displays a shape of the blood cell in the blood flow image of the frame of which the agglutination of the blood cell is detected and a shape of the blood cell of a previous frame of the aforementioned frame.

4. The blood cell trajectory displaying apparatus of claim 1,
wherein the analysis device calculates a change of area of the blood cell along the trajectory of the blood cell to a position of the agglutination by calculating an area of the blood cell in the blood flow image of the frame of which the agglutination of the blood cell is detected and an area of the blood cell of a previous frame of the aforementioned frame, when the analysis device detects the agglutination of the blood cell.

5. The blood cell trajectory displaying apparatus of claim 1,
wherein the analysis device calculates at least one of a movement speed and a movement angle between frames of the blood cell in the blood flow image of the frame of which the agglutination of the blood cell is detected and a previous frame of the aforementioned frame, when analysis device detects the agglutination of the blood cell.

6. The blood cell trajectory displaying apparatus of claim 5,
wherein the analysis device for determining that the blood cell is abnormal when at least one of the calculated movement speed and the movement angle exceeds predetermined ranges; and
the display device emphasizes and displays the trajectory of the blood cell, when the blood cell is determined as an abnormal by the analysis device more than the trajectory of the blood cell which is not determined as an abnormal.
